(19) United States
(12) Patent Application Publication
(10) Pub. No.: US 2026/0259094 A1
(43) Pub. Date: Sep. 3, 2026

(54) FLEXIBLE PRESSURE SENSORS, CONDUCTIVE TRANSFERS, AND METHODS OF MANUFACTURE FOR SUCH

(71) Applicant: INFI-TEX LTD, London (GB)

(72) Inventors: Nick RAWCLIFFE, London (GB); Jacob JELEN, London (GB); Andrew AUSTIN, London (GB); Cyril HILSUM, London (GB)

(21) Appl. No.: 19/165,084

(22) PCT Filed: Feb. 20, 2024
(86) PCT No.: PCT/GB2024/050472

§ 371 Date: Sep. 12, 2025

(30) Foreign Application Priority Data
Mar. 14, 2023 (GB) 2303704.7

(51) Int. Cl. G01L 5/102; G01L 1/22

(52) U.S. Cl. CPC G01L 5/102; G01L 1/2287

(57) ABSTRACT

An aspect of the invention relates to a flexible pressure sensor comprising a first electrode layer, a second electrode layer, a pressure sensing layer, and a spacer layer. The pressure sensing layer is positioned between the first electrode layer and the second electrode layer and comprises a pressure sensing material. The spacer layer is also positioned between the first electrode layer and the second electrode layer and is configured to separate the pressure sensing layer and the second electrode layer. Methods of manufacturing such a flexible sensor are also provided, including methods of manufacturing intermediate conductive transfers and laminates for assembly into the flexible sensor.

### FIELD OF THE INVENTION

The present disclosure relates to flexible pressure sensors and methods of manufacturing for such, in particular flexible textile pressure sensors comprising a spacer layer.

### BACKGROUND

Flexible pressure sensors, in particular textile pressure sensors, are key to the development of flexible electronics and have numerous applications across a broad range of industries. Such applications include, but are not limited to, flexible human-computer interfaces, wearable electronics, smart medical sensors, and Internet-of-Things sensors. However, manufacturing challenges exist in the production of such flexible pressure sensors. In particular, flexible pressure sensors rely on a small airgap between two sensor layers. Under the application of pressure, the sensor is deformed to close the air gap and contact the two sensor layers, resulting in a pressure measurement signal. Thus, if the two sensor areas are glued together, the sensor will no longer function.

Despite this, it is important that the two sensor layers remain properly aligned during use to maintain their functionality and consistency of response. The flexible sensors must also be sufficiently robust to withstand flexure and repeated application of pressure without suffering a loss of performance. Furthermore, the sensors should also be sufficiently robust to survive repeated washing and standard wear-and-tear.

Current manufacturing practice is to bond the area around the sensor. However, this can cause problems, particularly for large sensor areas, as the top sensor layer can collapse onto the bottom layer due to the flexible nature of the sensor. This can therefore cause the two sensor layers to contact and result in parasitic noise and erroneous pressure measurements.

There therefore remains a need to improve existing flexible pressure sensors and manufacturing methods for such to improve sensor performance, reliability, and enable them to be used in more complex applications, and at scale.

### SUMMARY OF THE INVENTION

Aspects of the invention are as set out in the independent claims and optional features are set out in the dependent claims. Aspects of the invention may be provided in conjunction with each other and features of one aspect may be applied to other aspects.

An aspect of the invention relates to a flexible pressure sensor comprising a first electrode layer, a second electrode layer, and a pressure sensing layer. The pressure sensing layer is positioned between the first electrode layer and the second electrode layer and comprises a pressure sensing material. The flexible pressure sensor further comprises a spacer layer positioned between the first electrode layer and the second electrode layer, wherein the spacer layer is configured to separate the pressure sensing layer and the second electrode layer. The spacer layer may advantageously reduce parasitic noise detected by the sensor by spacing apart the pressure sensing layer and the second electrode, thus reducing unintentional contact between the two layers in the absence of applied force (i.e., avoiding a zero-pressure response). Parasitic noise may be particularly problematic for large sensor areas. The spacer layer may also advantageously improve the useable lifetime of the sensor by reducing abrasion between the pressure sensing layer and the second electrode, and thus reducing wear of the pressure sensing layer.

The depth of the spacer layer may be configured to provide an air gap between the pressure sensing layer and the second electrode layer. This may be advantageous to maintain the functionality of the sensor, in particular for large sensors, by preventing collapse of the second electrode layer onto the pressure sensing layer, caused in part due to the flexible nature of the sensor and the large sensor area. This therefore reduces parasitic noise and erroneous pressure measurements which results from unintentional contact between the second electrode layer and the pressure sensing layer.

The pressure sensing material may be configured to vary its resistance as a function of applied force or pressure. For example, in the absence of pressure or applied force, the pressure sensing material may have a relatively high resistance, however upon application of pressure or force, the pressure sensing material may be configured to reduce its resistance in the direction of the applied force, for example in the z-direction. As such, the magnitude of force or pressure applied to the flexible sensor may be derivable based on the resistance measured across the pressure sensing material by the first and second electrode. As an example, the pressure sensing material may comprise particles of inorganic chalcogenide, such as a metal sulphide, dispersed in an insulator, such as a polymer like elastomer or polyurethane, for example as disclosed by patent application GB2585349A, “Force or pressure sensing composite material”. However, the skilled person will understand that any other suitable pressure sensing material, and in particular any other suitable pressure sensing inks, may be used.

The flexible pressure sensor may further comprise a first flexible substrate and a second flexible substrate, wherein the first electrode layer and the second electrode layer are positioned between the first flexible substrate and the second flexible substrate. At least one of the first flexible substrate and the second flexible substrate may be a textile layer, preferably wherein both the first flexible substrate and the second flexible substrate consist of a textile layer. The textile layer may include any textile, for example but not limited to elastic, non-elastic, waterproof, knitted, woven, non-woven, synthetic, or natural.

The flexible pressure sensor may further comprise a first non-conductive layer and a second non-conductive layer, the first non-conductive layer and the second non-conductive layer being positioned between the first flexible substrate and the second flexible substrate such that the first electrode layer and the second electrode layer are positioned between the first non-conductive layer and the second non-conductive layer. This may be advantageous to electrically insulate the outward facing surfaces of the first and second electrodes, for example to minimise external interference and noise in the pressure sensing functionality, for example wherein the pressure sensing functionality may be determined as a function of resistance through the pressure sensing layer.

The flexible pressure sensor may further comprise a first shielding layer and a second shielding layer, wherein the first shielding layer is positioned between the first flexible substrate and the first non-conductive layer, and the second shielding layer is positioned between the second flexible substrate and the second non-conductive layer. The first shielding layer and the second shielding layer are configured to minimise electromagnetic interference, for example by providing a Faraday cage or Faraday shield. Preferably, the first and second shielding layers may each comprise a flexible conductive mesh, wherein the mesh is printed using conductive ink.

The spacer layer may be positioned at least partially in a plane of the pressure sensing layer. This may be advantageous to minimise interaction between the pressure sensing layer and the spacer layer. Alternatively, the spacer layer may be positioned on top of the pressure sensing layer. This may be advantageous for ease of manufacture, wherein the spacer layer may simply be deposited onto the pressure sensing layer.

The spacer layer may have a depth greater than the depth of the pressure sensing layer. This may be advantageous to enable the spacer layer to provide an air gap between the pressure sensing layer and the second electrode layer, particularly in embodiments where the spacer layer is positioned at least partially in a plane of the pressure sensing layer.

The spacer layer may be a non-conductive material. This may be advantageous such that the spacer layer does not interact with the sensing functionality, for example by altering the resistance, between the first and second electrodes.

The spacer layer may be resiliently deformable, for example wherein the spacer layer material is elastic, such that the spacer layer is configured to be compressed under pressure. This may be advantageous to facilitate good electrical contact between the second electrode layer and the pressure sensing layer under pressure, reducing any measurement artifacts, including “dead zones” resulting from the spacer layer. In some examples, the spacer layer may comprise rubber.

The spacer layer may be non-continuous, such that the first electrode, the pressure sensing layer, and the second electrode are configured to electrically interact upon application of force or pressure to the flexible pressure sensor. The skilled person will understand “non-continuous” to mean a layer bound by a periphery, the layer comprising one or more gaps or voids, wherein the gaps or voids do not comprise the material of the layer. The non-continuous spacer layer may comprise a plurality of apertures. For example, the non-continuous spacer layer comprising a plurality of apertures may be a mesh, for example wherein the mesh may be woven, knitted, printed, or otherwise. The plurality of apertures may be advantageous to allow the first electrode, the pressure sensing layer, and the second electrode to electrically interact through the apertures upon application of force or pressure to the flexible pressure sensor. Aperture size may be proportional to the pressure sensitivity of the sensor, for example wherein larger apertures correspond to a higher degree of sensitivity to detect pressure. The skilled person will understand that the apertures are not limited to circular apertures, and may include, but are not limited to, square, rectangular, oval, triangular, or non-uniform apertures. Aperture size may be defined by at least one of the total area of the aperture, diameter of the aperture, or radius of the aperture.

Alternatively, the non-continuous spacer layer may comprise a plurality of discrete spacer structures, otherwise referred to as spacer elements. The plurality of spacer structures may be advantageous to allow the first electrode, the pressure sensing layer, and the second electrode to electrically interact in areas between or surrounding the plurality of spacer structures upon application of force or pressure to the flexible pressure sensor. The relative spacing between spacer structures may be proportional to the pressure sensitivity of the sensor, for example wherein larger spacing between spacer structures corresponds to a higher degree of sensitivity to detect pressure.

The skilled person will understand that the aperture size or spacing between spacer structures need not be homogeneous and can be varied across the spacer layer to vary the pressure sensitivity across the sensor. This may be advantageous to tune sensitivity of different regions of the sensor, for example wherein different regions of the sensor may require different pressure sensitivity configured for their application. For example, the spacer layer may act as a mask to reduce the area of the pressure sensing layer which interacts with the second electrode layer under the application of pressure. As such, purely for illustration, a region of a sensor expected to be exposed to high pressure in its application may have a larger area of spacer layer material in this region, relative to a different region of the sensor expected to be exposed to lower pressure and needing higher sensitivity. This may allow a single high sensitivity pressure sensing layer to detect a wide range of pressures, without overloading the electronic signal due to the larger masking effect of the spacer layer in regions of expected high pressure. The skilled person will understand that a larger area of spacer material may correlate to at least one of (i) smaller aperture size; (ii) fewer apertures per unit area, (iii) smaller spacing between spacer structures, and (iii) a higher density of spacer structures per unit area.

In some examples, the pressure sensing layer may be deposited and/or printed according to a first design, and the spacer layer may be deposited and/or printed according to a second design. A design may comprise an arrangement or layout of material. Optionally, the first design of pressure sensing material and the second design do not overlap. This may be advantageous to ensure that the spacer layer does not interact with the pressure sensing layer which may otherwise cause anomalous pressure sensing readings in the region of overlap or interaction between the spacer layer and pressure sensing layer.

The first design of the pressure sensing layer may comprise at least one region of pressure sensing material, wherein the pressure sensing layer also comprises at least one void region without pressure sensing material, for example wherein the pressure sensing layer is non-continuous. In such examples, the spacer layer may comprise plurality of spacer structures, wherein at least one spacer structure of the plurality of spacer structures is bound within the at least one void region. Optionally, at least a portion of the spacer layer may additionally be arranged adjacent to at least a portion of a periphery of the pressure sensing layer.

The spacer layer may comprise an adhesive configured to adhere the spacer layer between the first electrode layer and the second electrode layer. In some examples, the spacer layer may consist of adhesive, for example an adhesive polymer. In other examples, the spacer material may be impregnated with or at least partially coated by an adhesive. For example, adhesive may be applied to spacer layer by dipping, spraying, pre-impregnating, printing, etc. Providing adhesive within the spacer layer may be advantageous to minimise relative movement and misalignment between the first electrode and the second electrode which may otherwise introduce noise and/or errors into the pressure sensing measurements and functionality of the flexible pressure sensor. The provision of adhesive may also improve the useable lifetime of the sensor by reducing relative movement between the pressure sensing layer and the second electrode, and thus reducing wear and abrasion of the pressure sensing layer.

The adhesive may be selectively activated, for example wherein the adhesive may be at least one of UV-curing, heat activated or thermosetting, or a multi-part adhesive. However, the skilled person will understand that any other adhesive or selectively activated adhesive may be used. This may be advantageous to facilitate printing the spacer layer from a single material, wherein the adhesive properties of the material may be selectively activated only in specific portions of the spacer layer, thereby avoiding the need to deposit an adhesive material separately to a spacer material, particularly in embodiments where adhesive is not desired across the entirety of the spacer layer.

In another aspect of the invention there is provided a method of producing a flexible pressure sensor, the method comprising obtaining a first flexible laminate and a second flexible laminate, wherein the first flexible laminate comprises a first flexible substrate, a first electrode layer, and a pressure sensing layer, and the second flexible laminate comprises a second flexible substrate and a second electrode layer. The pressure sensing layer is arranged on a first side of the first electrode layer such that pressure sensing layer is arranged on the side of the first electrode layer opposite to the side of the electrode layer adjacent to the first flexible substrate. The second electrode layer also comprises a first side opposite to the second flexible substrate and a second side adjacent to the flexible substrate.

The method further comprises depositing a non-conductive material onto at least one of: (i) the first side of the first electrode layer, (ii) the first side of the second electrode layer, or (iii) the pressure sensing layer, to form a spacer layer. The method further comprises attaching the first flexible laminate to the second flexible laminate, such that the pressure sensing layer and the second electrode layer are separated by the spacer layer.

Incorporating a spacer layer into the resulting flexible pressure sensor may advantageously improve the useable lifetime of the sensor by reducing wear of the pressure sensing layer caused by glancing abrasion between the pressure sensing layer and the second electrode. The spacer layer may also advantageously reduce parasitic noise detected by the sensor, for example wherein the parasitic noise is caused by the upper layers of the sensor pressing onto the pressure sensing layer. Parasitic noise may be particularly problematic for large sensor areas.

The skilled person will understand the method disclosed herein may be configured produce the flexible pressure sensor of the first aspect of the invention.

Depositing the non-conductive material to form the spacer layer may comprise printing the non-conductive material. Alternatively, depositing the non-conductive material to form a space layer may comprise depositing a prefabricated non-conductive, non-continuous layer, for example such as a mesh or other prefabricated non-conductive layer of material comprising a plurality of apertures. The depth of the spacer layer may be greater than the depth of the pressure sensing layer.

In some examples, obtaining the first flexible laminate may comprise fabricating the first flexible laminate, for example using a bottom-up approach, such as a direct printing method. This may comprise (i) printing a non-conductive material onto the flexible substrate to produce a non-conductive layer; (ii) printing an electrically conductive material onto the non-conductive layer to form the first electrode layer; and (iii) printing a pressure sensing material onto the first electrode layer to form the pressure sensing layer. The pressure sensing material may be a pressure sensing ink. The electrically conductive material may be an electrically conductive ink.

Alternatively, obtaining the first flexible laminate may comprise fabricating the first flexible laminate, for example using a top-down approach, such as a conductive transfer method. This may comprise (i) printing pressure sensing material onto a transfer substrate to produce the pressure sensing layer; (ii) printing an electrically conductive material onto the pressure sensing layer to produce the first electrode layer; (iii) printing a non-conductive material over the first electrode layer to produce a first non-conductive layer; and (iv) printing an adhesive material over the non-conductive layer to produce a transfer adhesive layer. The method may then further comprise attaching the transfer adhesive layer to the first flexible substrate; and removing the transfer substrate from the pressure sensing layer. As above, the pressure sensing material may be a pressure sensing ink, and/or the electrically conductive material may be an electrically conductive ink.

In some examples, printing the pressure sensing material to produce the pressure sensing layer may comprise printing the pressure sensing material according to a first design, and depositing the non-conductive material to form the spacer layer may comprise printing the non-conductive spacer material according to a second design, wherein a design may comprise an arrangement or layout of material. Optionally, the first design of pressure sensing material and the second design do not overlap. For example, the first design of the pressure sensing layer may comprise at least one region of pressure sensing material, wherein the pressure sensing layer also comprises at least one void region without pressure sensing material. In such examples, a plurality of spacer structures constituting the spacer layer may be printed within the at least one void region. Optionally, at least a portion of the spacer layer may additionally be printed adjacent to at least a portion of a periphery of the pressure sensing layer.

The non-conductive material of the spacer layer may be an adhesive material, such that the spacer layer is configured to provide a first adhesive layer. The first flexible laminate and the second flexible laminate may then be coupled together by the first adhesive layer. Providing the first adhesive layer to attach the first flexible laminate to the second flexible laminate may be advantageous to minimise relative movement and misalignment between the first electrode and the second electrode which may otherwise introduce noise and/or errors into the pressure sensing measurements and functionality of the resulting flexible pressure sensor. The provision of adhesive may also improve the useable lifetime of the sensor by reducing wear of the pressure sensing layer caused by relative movement between the pressure sensing layer and the second electrode.

In some examples, the non-conductive material of the spacer layer may be a thermosetting adhesive material, wherein the method may further comprise applying targeted heat patterning, for example but not limited to using a targeted heat press plate. Applying targeted heat patterning may be configured to apply heat to only a portion of the flexible sensor, such that only a portion of the spacer layer is configured to form the first adhesive layer. As such, only the portion of the spacer layer which forms the first adhesive layer is configured to couple together the first flexible substrate and the second flexible substrate.

The skilled person will understand this may be applied mutatis mutandis for other selectively activated adhesives. For example, the non-conductive material of the spacer layer may be a UV-curing adhesive material, wherein the method may further comprise applying UV light patterning. Applying UV light patterning may be configured to apply UV light to only a portion of the spacer layer such that only a portion of the spacer layer is configured to form the first adhesive layer. As such, only the portion of the spacer layer which forms the first adhesive layer is configured to couple together the first flexible substrate and the second flexible substrate.

Alternatively, the method may further comprise printing an adhesive material onto at least one of (i) the first side of the first electrode layer, (ii) the first side of the second electrode layer, (iii) the pressure sensing layer, or (iv) the spacer layer, to form the first adhesive layer. As above, the first adhesive layer is configured to couple together the first flexible laminate and the second flexible laminate. The depth of the first adhesive layer may be greater than or equal to the depth of the pressure sensing layer.

In some examples wherein the pressure sensing material is printed according to a first design, printing the first adhesive layer comprises printing a third design in the adhesive material, wherein the first design of the pressure sensing material and the third design of adhesive material do not overlap.

In another aspect of the invention there is provided a conductive transfer for application to an article. The conductive transfer comprises a pressure sensing layer comprising a pressure sensing material according to a first design and a first adhesive layer positioned at least partially in a plane of the pressure sensing layer, wherein the first adhesive layer comprises an adhesive material according to a second design, wherein the first design and the second design do not overlap.

The conductive transfer further comprises a first electrode layer positioned adjacent to the pressure sensing layer and the first adhesive layer, a first non-conductive layer positioned adjacent to a side of the first electrode layer opposite to the pressure sensing layer and first adhesive layer, and a second adhesive layer (as referred to as a transfer adhesive layer) positioned adjacent to a side of the first non-conductive layer opposite to the first electrode layer, configured to adhere the conductive transfer to the article.

Providing a first adhesive layer positioned at least partially in a plane of the pressure sensing layer, wherein the first design of the pressure sensing material and the second design of the adhesive do not overlap may be advantageous to adhere the transfer to a second flexible laminate to form the sensor. Providing the adhesive in a non-overlapping design allows the adhesive to be provided in the sensing area, without interfering with the pressure sensing functionality of the pressure sensing layer. Providing the adhesive may also be advantageous to minimise relative movement and misalignment between the first electrode layer and the second electrode layer once assembled into a sensor, which may otherwise introduce noise and/or errors into the pressure sensing measurements and functionality of the resulting flexible pressure sensor. The provision of adhesive may also improve the useable lifetime of the sensor by reducing wear of the pressure sensing layer caused by relative movement between the pressure sensing layer and the second electrode.

In some examples, the first design of the pressure sensing material comprises at least one region of pressure sensing material bound by a periphery. The pressure sensing layer may also comprise at least one void region without pressure sensing material inside the periphery, wherein at least a portion of the first adhesive layer is bound within the at least one void region in accordance with a second design. Optionally, at least a portion of the first adhesive layer may be arranged adjacent to at least a portion of a periphery of the pressure sensing layer, such that the first design of the pressure sensing material and the second design of the adhesive material do not overlap.

The skilled person will understand that the conductive transfer as discussed above may be configured for use in the flexible pressure sensor of the previous aspect. For example, the conductive transfer may be configured for application to a first flexible substrate, for example wherein the first flexible substrate may be a textile.

The pressure sensing layer and first adhesive layer may be printed onto a removable substrate. This may be advantageous such that the removable substrate is configured to be removed from the conductive transfer such that the first adhesive layer can then subsequently be exposed and attached to the final article at a later stage or in situ. For example, wherein the first adhesive layer is configured to attach to a separate electrode, for example as part of a separate conductive transfer, to assemble a flexible sensor within the scope of the first aspect of the invention.

The depth of the first adhesive layer may be equal to or less than the depth of the pressure sensing layer. This may be advantageous to avoid warping of the adjacent layer in the conductive transfer. Alternatively, the depth of the first adhesive layer may be greater than the depth of the pressure sensing layer such that the first adhesive layer is configured to provide a spacer layer.

There is also provided a method of producing a conductive transfer for application to an article. The skilled person will understand the method may be suitable for producing the conductive transfer of the preceding aspect of the invention. The method comprises printing a pressure sensing material onto a transfer substrate to produce a pressure sensing layer, printing a first adhesive material onto the transfer substrate to produce a first adhesive layer, printing an electrically conductive ink onto the pressure sensing layer and the first adhesive layer to form an electrode layer, printing a non-conductive ink over the electrode layer to produce a non-conductive layer, and printing a second adhesive material over the non-conductive layer to produce a second adhesive layer (otherwise referred to as a transfer adhesive layer). Printing the pressure sensing layer comprises printing a first design in the pressure sensing material and printing the first adhesive layer comprises printing a second design in the first adhesive material, wherein the first design and the second design do not overlap.

In some examples, printing the pressure sensing layer (i.e., the first design) may comprise printing at least one region of pressure sensing material bound by a periphery, and at least one void region without pressure sensing material within the periphery, wherein printing the first adhesive layer (i.e., the second design) comprises printing at least a portion of the adhesive material within the at least one void region, such that the first design of the pressure sensing material and the second design of the adhesive material do not overlap. Additionally, or instead, at least a portion of the first adhesive material may be printed adjacent to at least a portion of a periphery of the pressure sensing layer (i.e., the first design), such that the first design of the pressure sensing material and the second design of the adhesive material do not overlap.

The method may further comprise adhering the second adhesive layer to the article, for example wherein the article comprises a flexible substrate, such as a textile.

The method may further comprise removing the first substrate from the pressure sensing layer and the first adhesive layer, for example wherein the first substrate is a transfer substrate, such as a transfer film.

The method may further comprise adhering the first adhesive layer to a second article, for example wherein the second article comprises a second electrode, for example wherein the second article comprises a second flexible laminate, such that the adhered article forms a flexible pressure sensor.

In another aspect of the invention there is provided a method of producing a flexible pressure sensor. The method comprises obtaining a first flexible laminate, wherein the first flexible laminate comprises a first flexible substrate, a first electrode layer, and a pressure sensing layer, the pressure sensing layer being arranged on a first side of the first electrode layer opposite to the flexible substrate. The method then comprises printing a non-conductive material onto the first side of the first electrode layer to produce a spacer layer.

Printing the spacer layer may comprise printing the non-conductive material according to a design, wherein the design of non-conductive material does not overlap with the pressure sensing material within the pressure sensing layer, wherein the pressure sensing material is printed according to a first design. The first design may be non-continuous. This may be advantageous to avoid interaction between the spacer layer and the pressure sensing material of the pressure sensing layer which may otherwise interfere with the pressure sensing functionality of the pressure sensing layer. Printing the spacer layer in a non-overlapping design to the pressure sensing material may result in the spacer layer being at least partially in a plane of the pressure sensing layer. However, in such examples, the depth of the spacer layer is greater than the depth of the pressure sensing layer.

Obtaining the first flexible laminate may comprise printing at least the first electrode layer and the pressure sensing layer onto the first flexible substrate, for example by a bottom-up fabrication approach. This may comprise printing a non-conductive ink onto the flexible substrate to produce a non-conductive layer, printing an electrically conductive ink onto the non-conductive layer to form the first electrode layer, and printing a pressure sensing ink onto the first electrode layer according to a first design to produce a pressure sensing layer, wherein printing the pressure sensing layer comprises printing the first design in the second electrically conductive ink. The first design may be a continuous layer, or a non-continuous layer. The electrically conductive ink of the first electrode layer is not a pressure sensing ink.

Alternatively, obtaining the first flexible substrate may comprise obtaining the first flexible substrate by means of a conductive transfer, for example using a top-down fabrication approach. This may comprise printing a pressure sensing ink onto a transfer substrate to produce the pressure sensing layer, printing an electrically conductive ink onto the pressure sensing layer to form the first electrode layer, and printing a non-conductive ink over the first electrode layer to produce a non-conductive layer. An adhesive material is then printed over the non-conductive layer to produce an adhesive layer. The adhesive layer is then attached to the first flexible substrate, and the transfer substrate is removed from the pressure sensing layer.

The non-conductive material of the spacer layer may be an adhesive material, such that the spacer layer is configured to be at least partially adhesive. In such examples, the spacer layer may also provide a first adhesive layer.

In some examples, the non-conductive material of the spacer layer may be a thermosetting adhesive material. As such, the method may further comprise applying targeted heat patterning to only a portion of the flexible sensor, such that only a portion of the spacer layer is configured to form the first adhesive layer, for example but not limited to using a targeted heat press plate. As a result, only the portion of the spacer layer which forms the first adhesive layer is configured to couple together the first flexible substrate and the second flexible substrate.

The skilled person will understand this may be applied mutatis mutandis for other selectively activated adhesives. For example, the non-conductive material of the spacer layer may be a UV-curing adhesive material, wherein the method may further comprise applying UV light patterning. Applying UV light patterning may be configured to apply UV light to only a portion of the spacer layer such that only a portion of the spacer layer is configured to form the first adhesive layer. As such, only the portion of the spacer layer which forms the first adhesive layer is configured to couple together the first flexible substrate and the second flexible substrate.

The method may further comprise printing an adhesive material onto the first side of the first electrode layer to form the first adhesive layer. The method may then further comprise attaching the first flexible laminate to a second flexible laminate via the first adhesive layer. In such examples, the second flexible laminate comprises an electrode layer, such that the electrode layer of the second flexible substrate forms a second electrode layer of the flexible sensor. Attaching the first adhesive layer to the second flexible laminate may comprise applying heat and/or pressure, for example by using a targeted heat press as described above. The depth of the first adhesive layer may be greater than or equal to the depth of the pressure sensing layer.

In some examples wherein the pressure sensing material is printed according to a first design, printing the first adhesive layer comprises printing a third design in the adhesive material, wherein the first design of the pressure sensing material and the third design of adhesive material do not overlap.

### DRAWINGS

Embodiments of the disclosure will now be described, by way of example only, with reference to the accompanying drawings, in which:

FIG. 1A shows an exploded view of an example embodiment of a flexible pressure sensor of the present invention.

FIG. 1B shows a cross-section of the example flexible pressure sensor of FIG. 1A.

FIG. 2A shows an exploded view of another example embodiment of a flexible pressure sensor of the present invention.

FIG. 2B shows a cross-section of the example flexible pressure sensor of FIG. 2A.

FIG. 3 is a flow diagram illustrating an example method of fabricating a flexible pressure sensor, such as the flexible pressure sensor of either of FIGS. 1A-1B, or FIGS. 2A-2B.

FIG. 4 shows an exploded view of an example conductive transfer, suitable for use in the method of FIG. 3 for fabricating a flexible pressure sensor, such as the flexible pressure sensor of either of FIGS. 1A-1B, or FIGS. 2A-2B.

FIG. 5A shows an exploded view of an example flexible laminate formed by direct printing, suitable for use in the method of FIG. 3 for fabricating a flexible pressure sensor, such as the flexible pressure sensor of either of FIGS. 1A-1B, or FIGS. 2A-2B.

FIG. 5B shows an exploded view of a complementary flexible laminate to the laminate of FIG. 5A, such that the combination of FIGS. 5A and 5B is suitable for fabricating a flexible pressure sensor, such as the flexible pressure sensor of either of FIGS. 1A-1B, or FIGS. 2A-2B.

FIG. 6A shows an exploded view of another example flexible laminate formed by direct printing, suitable for use in the method of FIG. 3 for fabricating a flexible pressure sensor, such as the flexible pressure sensor of either of FIGS. 1A-1B, or FIGS. 2A-2B.

FIG. 6B shows an exploded view of a complementary flexible laminate to the laminate of FIG. 6A, such that the combination of FIGS. 6A and 6B is suitable for fabricating a flexible pressure sensor, such as the flexible pressure sensor of either of FIGS. 1A-1B, or FIGS. 2A-2B.

FIG. 7 shows a cross-section of an example conductive transfer comprising a spacer layer.

FIG. 8 shows an exploded view of another example conductive transfer comprising an additional adhesive layer, suitable for fabricating a flexible sensor.

FIG. 9A shows an exploded view of an alternative flexible laminate formed by direct printing, suitable for use in the method of FIG. 3 for fabricating a flexible pressure sensor, such as the flexible pressure sensor of either of FIGS. 1A-1B, or FIGS. 2A-2B, wherein the flexible laminate comprises an additional adhesive layer and a spacer layer.

FIG. 9B shows an exploded view of a complementary flexible laminate to the laminate of FIG. 9A, such that the combination of FIGS. 9A and 9B is suitable for fabricating a flexible pressure sensor, such as the flexible pressure sensor of either of FIGS. 1A-1B, or FIGS. 2A-2B.

FIG. 10 shows a top view of an example configuration wherein a first flexible laminate, such as the laminate of FIG. 5A or 9A, and a second flexible laminate, such as the laminate of FIG. 5B or 9B, are deposited onto a single flexible substrate on opposite sides of a fold line.

FIG. 11 shows a top view of an example flexible laminate, for example such as the flexible laminate of FIG. 5A, for use in a flexible pressure sensor, such as the flexible pressure sensor of FIGS. 1A-1B.

FIG. 12A shows a top view of another example flexible laminate for use in a flexible pressure sensor, such as the flexible pressure sensor of FIGS. 1A-1B.

FIG. 12B shows the flexible laminate of FIG. 12A being adhered to a second flexible laminate using a targeted heat press, for example in accordance with the method of FIG. 3.

### SPECIFIC DESCRIPTION

Embodiments of the claims relate to flexible pressure sensors, methods of manufacture and conductive transfers for such.

It will be appreciated from the discussion above that the embodiments shown in the Figures are merely exemplary, and include features which may be generalised, removed or replaced as described herein and as set out in the claims.

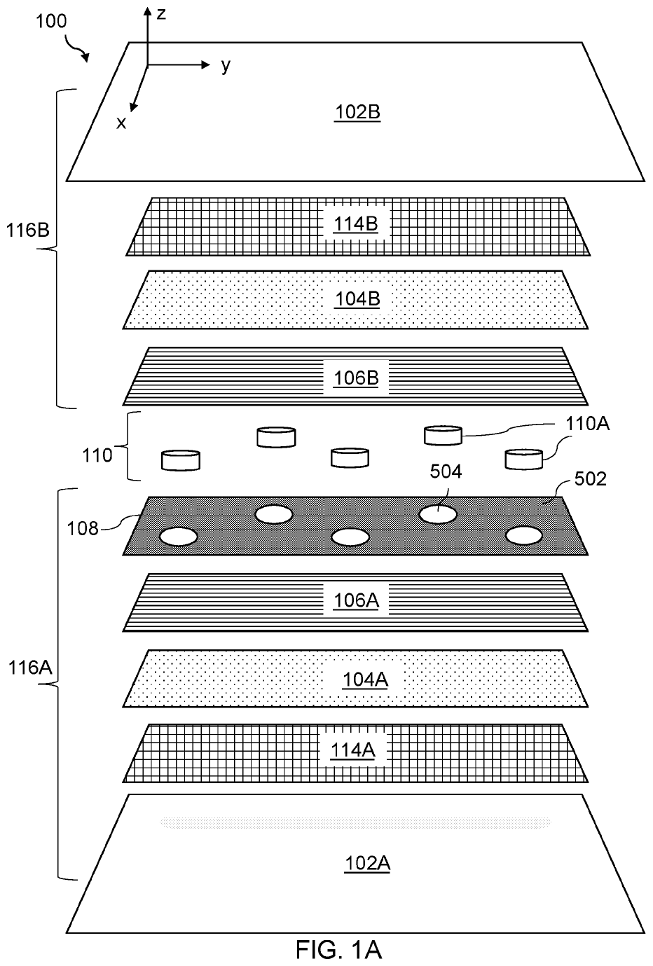

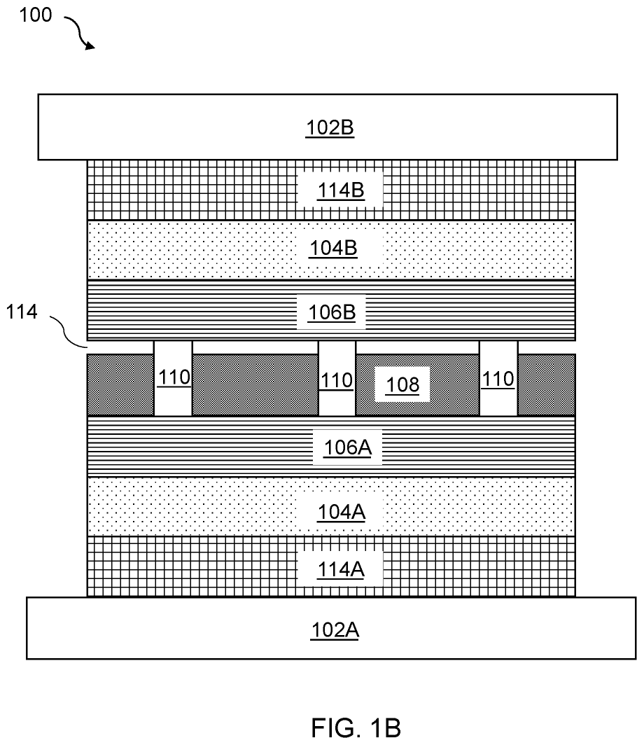

FIG. 1A illustrates an exploded view of an example embodiment of a flexible pressure sensor 100 of the present invention. FIG. 1B shows a cross-sectional view of the same flexible pressure sensor 100.

The flexible pressure sensor 100 comprises a first flexible substrate 102A and a second flexible substrate 102B. In this example, the first flexible substrate 102A and the second flexible substrate 102B are textiles.

The flexible pressure sensor 100 also comprises a first non-conducting layer 104A and a second non-conducting layer 104B arranged in between the first flexible substrate 102A and the second flexible substrate 102B. The first non-conducting layer 104A and the second non-conducting layer 104B are configured to be electrically insulating.

The flexible pressure sensor 100 further comprises a first electrode layer 106A and a second electrode layer 106B arranged in between the first non-conducting layer 104A and the second non-conducting layer 104B such that the first electrode layer 106A is adjacent to the first non-conducting layer 104A and the second electrode layer 106B is adjacent to the second non-conducting layer 104B. Each of the first and second electrode layers 106A and 106B comprises an electrically conductive ink printed according to an electrode design (not shown). The skilled person will understand that the electrode design of the second electrode layer 106B may be the same or different to the electrode design of the first electrode layer 106A. The electrode designs of the first and second electrode layers 106A and 106B are configured to provide localised electrical resistance sensing functionality across the pressure sensing layer 108 by providing a plurality of sensing points across the electrode layers 106A and 106B. The plurality of sensing points are configured such that pressure applied to the surface of the sensor 100 may be resolved in three-dimensional space, including x, y, and z directions. As an example, the electrode designs of the first and second electrode layers, 106A and 106B, may comprise an array of four-terminal sensing elements.

The flexible sensor 100 further comprises a pressure sensing layer 108 arranged between the first electrode layer 106A and the second electrode layer 106B. The pressure sensing layer 108 consists of a pressure sensing material. In this example, the pressure sensing material is a pressure sensing ink configured to vary its resistance as a function of applied force or pressure. As an example, the pressure sensing ink may comprise particles of inorganic chalcogenide, such as a metal sulphide, dispersed in an insulator, such as a polymer like elastomer or polyurethane, for example as disclosed by patent application GB2585349A, “Force or pressure sensing composite material”. However, the skilled person will understand that any other suitable pressure sensing material may be used.

The flexible sensor 100 also comprises a non-continuous spacer layer 110 positioned between the first electrode layer 106A and the second electrode layer 106B. The spacer layer 110 is an electrically insulating material. The spacer layer is also a resiliently deformable material, such as a rubber or other elastic polymer.

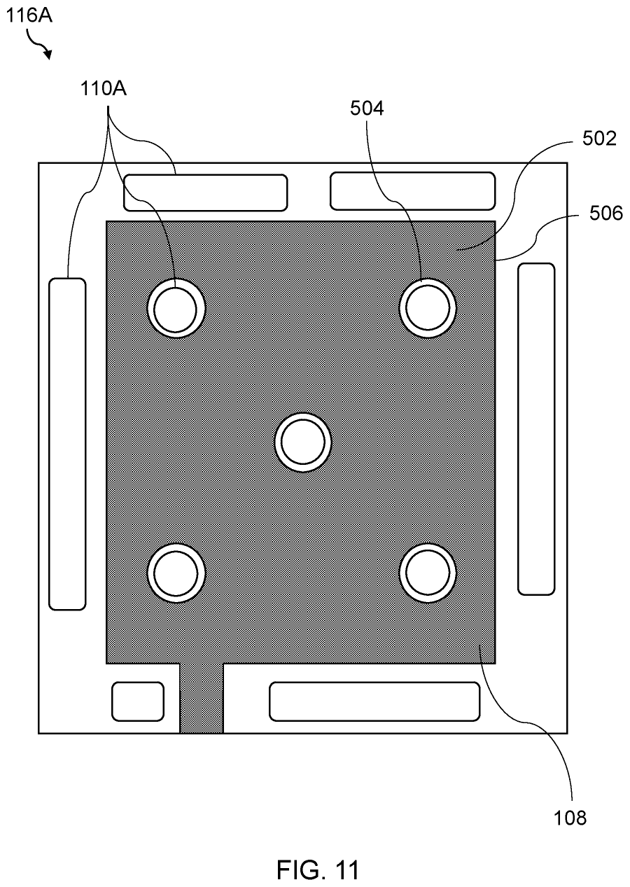

In the example shown in FIGS. 1A and 1B, the pressure sensing layer 108 is printed according to a first design and the spacer layer 110 is printed according to a second design, comprising a plurality of spacer structures 110A, wherein the first design and the second design do not overlap. In the example shown, the first design of the pressure sensing layer 108 comprises a region of pressure sensing material 502, and a plurality of distributed void regions 504 within the region of pressure sensing material 502, without pressure sensing material. In the example shown, the plurality of spacer structures 110A are arranged within void regions 504. Optionally, in addition, at least a portion of the spacer layer 110 may be arranged adjacent to at least a portion of a periphery of the pressure sensing layer as shown in more detail in FIG. 11.

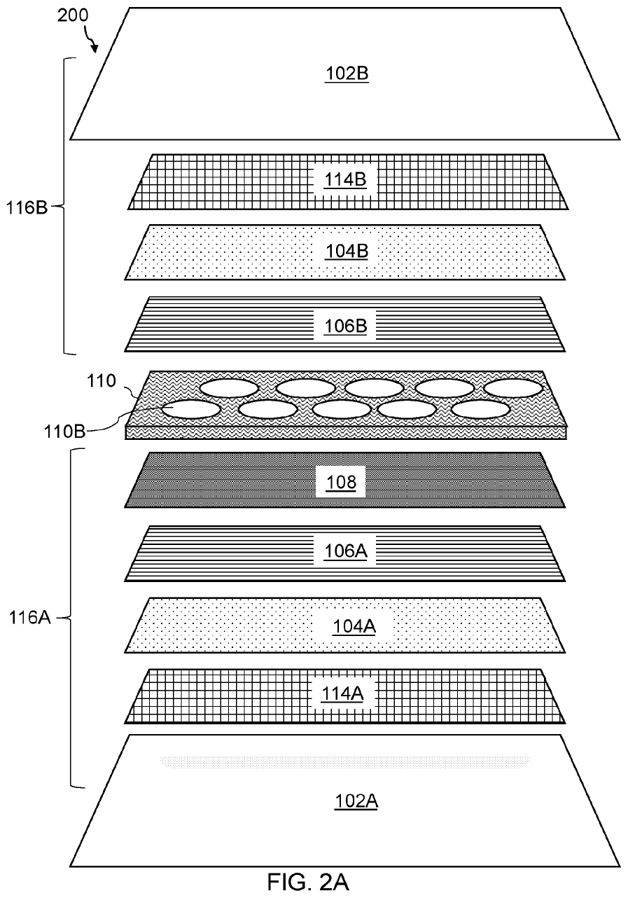

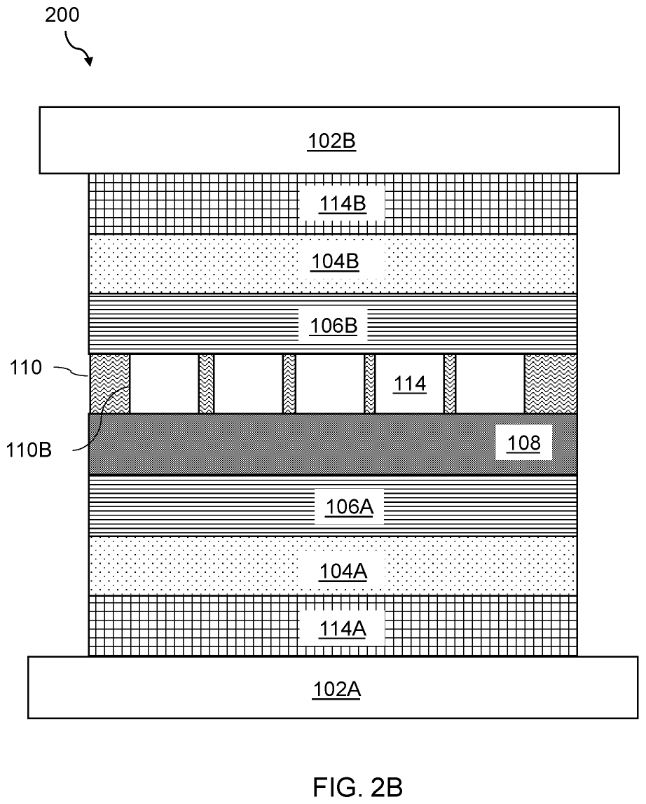

As shown in FIG. 1B, the spacer layer 110 is positioned partially in a plane common to the pressure sensing layer 108, however, the spacer layer 110 has a thickness (or “depth”) greater than that of the pressure sensing layer 108. The skilled person will understand, however, that in other examples, the spacer layer 110 and the pressure sensing layer 108 may not be arranged within a common plane, for example as illustrated by the embodiment shown in FIGS. 2A-2B. The skilled person will also understand that in other examples, the spacer layer 110 and the pressure sensing layer 108 may overlap. For example, in an alternative embodiment the pressure sensing layer 108 may be continuous, wherein the plurality of spacer structures 110A are arranged on top of the pressure sensing layer 108.

Optionally, as shown in FIGS. 1A and 1B, the flexible sensor 100 further comprises a first shielding layer 114A and a second shielding layer 114A, each configured to be electrically conductive. In this example, each of the shielding layers 114A and 114B consists of a printed mesh of electrically conductive ink. The shielding layers 114A and 114B are configured to block electromagnetic fields external to the flexible pressure sensor by providing a Faraday cage.

The spacer layer 110 is configured to separate the pressure sensing layer 108 and the second electrode layer 106B in the absence of pressure or applied force to the sensor 100. The depth of the spacer layer 110 is configured to provide an air gap 114 between the pressure sensing layer 108 and the second electrode layer 106B such that an air gap insulates the second electrode 106B from the pressure sensing layer 108 in the absence of pressure or applied force.

In use, as pressure is applied to a region of the sensor 100, the second flexible substrate 102B deforms such that the second electrode 106B contacts the pressure sensing layer 108 in between the spacer structures 110A in the region of applied pressure, closing the air gap 114. This completes an electrical circuit between the first electrode 106A and the second electrode 106B, via the pressure sensing layer 108. The magnitude of the pressure applied is determined as a function of the resistivity of the pressure sensing layer 108.

In the absence of applied pressure, the pressure sensing ink in the pressure sensing layer 108 has a relatively high resistance. However, upon application of pressure or force, the pressure sensing ink is configured to reduce its resistance in the direction of the applied force proportionally to the magnitude of force or pressure applied.

The first and second electrode layers 106A and 106B sense the electrical resistance at an array of points across the pressure sensing layer 108. As such, the magnitude of force or pressure applied at each point across pressure sensing layer is derivable based on the resistance measured across the pressure sensing layer by the first and second electrodes, 106A and 106B.

Upon removal of the applied force or pressure, the flexible sensor relaxes such that the second electrode layer 106B is once again separated from the pressure sensing layer 108 by the spacer layer 110 such that an air gap insulates the second electrode 106B from the pressure sensing layer 108.

FIG. 2A shows an exploded view of another example embodiment of a flexible pressure sensor 200 of the present invention. FIG. 2B shows a cross-section of the example flexible pressure sensor 200 of FIG. 2A.

The flexible sensor 200 has substantially the same structure as the flexible sensor 100 described in relation to FIGS. 1A and 1B. However, the flexible sensor 200 of FIGS. 2A and 2B comprises a non-continuous spacer layer 110 comprising a plurality of apertures 110B, instead of a plurality of discrete spacer elements 110A. In the example shown in FIG. 2A, the non-continuous spacer layer 110 comprises a mesh of printed electrically insulating material, the mesh comprising the plurality of apertures 110B. Preferably, the mesh is also resiliently deformable.

The skilled person will understand that the aperture size and/or spacing between apertures 110B need not be homogeneous and can be varied across the spacer layer 110 to vary the pressure sensitivity across the sensor 200. This may be advantageous to tune sensitivity of different regions of the sensor 200, for example wherein different regions of the sensor may require different pressure sensitivity configured for their application.

The spacer layer 110 is arranged between the pressure sensing layer 108 and the second electrode layer 106B. The pressure sensing layer 108 consists of a continuous layer of pressure sensing ink. As such, the spacer layer 110 in arranged adjacent to the pressure sensing layer 108.

As described in relation to FIGS. 1A and 1B, the spacer layer 110 is configured to separate the pressure sensing layer 108 and the second electrode layer 106B in the absence of pressure or applied force to the sensor 100. The depth of the spacer layer 110 is configured to provide an air gap 114 between the pressure sensing layer 108 and the second electrode layer 106B in the absence of pressure or applied force, wherein the air gap 114 is provided within the apertures 110B of the spacer layer 110.

As pressure is applied to a region of the sensor 100 in use, the second flexible substrate 102B deforms such that the second electrode 106B contacts the pressure sensing layer 108 through at least one of the plurality of apertures 106B in the region of applied pressure, displacing the air within the aperture 114. This completes an electrical circuit between the first electrode 106A and the second electrode 106B, via the pressure sensing layer 108.

The first and second electrode layers 106A and 106B then sense the electrical resistance across the pressure sensing layer 108 based on the resistance at each of the plurality of sensing points. As such, the magnitude of force or pressure applied to the sensor 200 is derivable based on the resistance measured across the pressure sensing layer by the first and second electrodes, 106A and 106B.

Upon removal of the applied force or pressure, the flexible sensor relaxes such that the second electrode layer 106B is once again separated from the pressure sensing layer 108 by the spacer layer 110 such that an air gap 114 within the apertures 110B insulates the second electrode 106B from the pressure sensing layer 108.

### Method of Manufacture

Both flexible pressure sensors 100 and 200 may be assembled from two parts, referred to as a first flexible laminate 116A and a second flexible laminate 116B. As shown in FIGS. 1A and 2A, the first flexible laminate 116A comprises the first flexible substrate 102A, the first shielding layer 114A, the first non-conducting layer 104A, the first electrode layer 106A, and the pressure sensing layer 108. The second flexible laminate 116B comprises the second flexible substrate 102B, the second shielding layer 114B, the second non-conducting layer 104B, and the second electrode layer 106B.

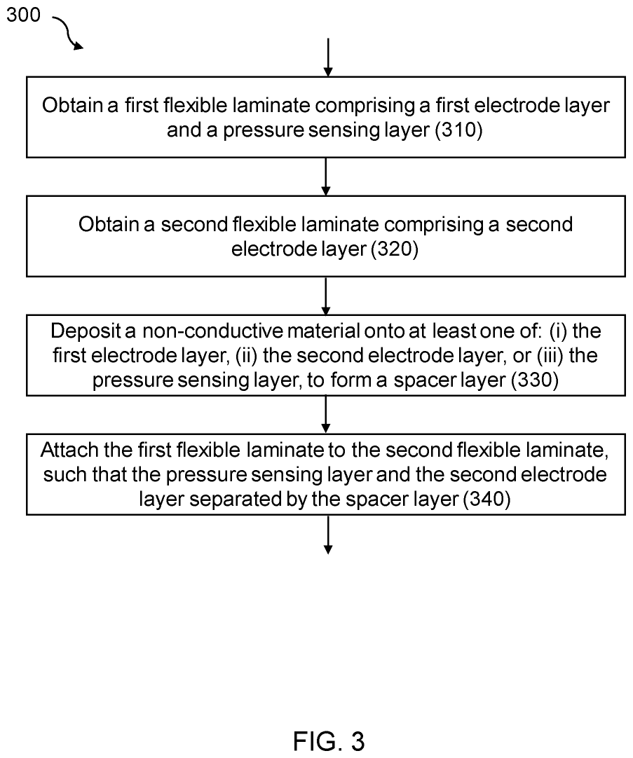

An example fabrication method 300 is illustrated by way of a flow chart in FIG. 3. Firstly, the method comprises obtaining a first flexible laminate 116A (310), wherein the first flexible laminate 116A comprises a first flexible substrate 102A, a first electrode layer 106A, and a pressure sensing layer 108. For the flexible sensor 100 of FIGS. 1A and 1B and sensor 300 of FIGS. 2A and 2B, the first flexible laminate 116A comprises the first flexible substrate 102A, the first shielding layer 114A, the first non-conducting layer 104A, the first electrode layer 106A, and the pressure sensing layer 108.

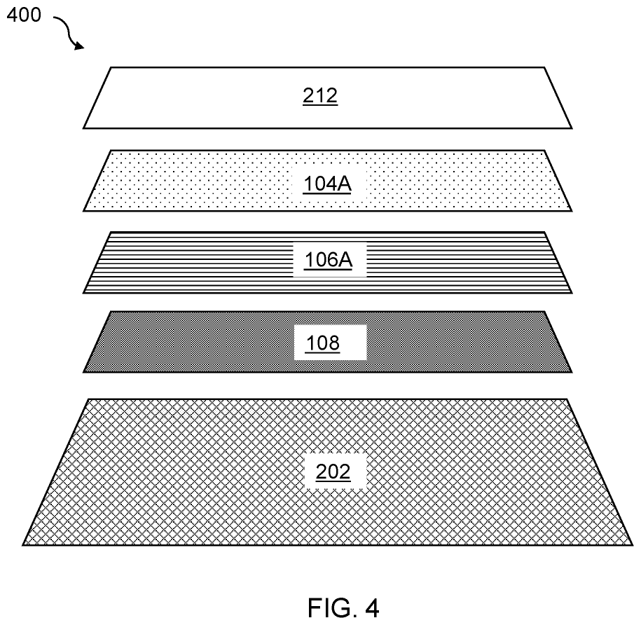

The first flexible laminate 116A may be obtained by any suitable method. As an example, the first flexible laminate 116A may be obtained by way of a conductive transfer, as shown in FIG. 4. In such examples, obtaining the first flexible laminate 116A comprises printing pressure sensing material, such as a pressure sensing ink, onto a transfer film 202 to produce the pressure sensing layer 108, printing an electrically conductive material, such as an electrically conductive ink, onto the pressure sensing layer 108 to form the first electrode layer 106A, printing a non-conductive material over the first electrode layer 106A to produce a non-conductive layer 104A, and printing an adhesive material over the non-conductive layer 104A to produce a transfer adhesive layer 212. The method may then further comprise attaching the transfer adhesive layer 212 to a first flexible substrate 102A and removing the transfer film 202 from the pressure sensing layer 108. The transfer adhesive 212 is configured to adhere the insulating layer 104A to the first flexible substrate 102A to form the first flexible laminate 116A.

The method 300 also comprises obtaining a second flexible laminate 116B (320), wherein the second flexible laminate 116B comprises at least a second electrode layer 106B. For the flexible sensor 100 of FIGS. 1A and 1B and the flexible sensor 200 of FIGS. 2A and 2B, the second flexible laminate 116B comprises the second flexible substrate 102B, the second shielding layer 114B, the second non-conducting layer 104B, and the second electrode layer 106B. As for the first flexible laminate 116A, the second flexible laminate 116B may be obtained by any suitable method. As an example, the second flexible laminate 116B may be obtained by way of a conductive transfer. In such examples, obtaining the second flexible laminate 116B comprises printing an electrically conductive material, such as an electrically conductive ink onto a transfer film 202 to form the second electrode layer 106B, printing a non-conductive material over the second electrode layer 106B to produce a non-conductive layer 104B, and printing an adhesive material over the non-conductive layer 104B to produce a transfer adhesive layer 212. The method may then further comprise attaching the transfer adhesive layer 212 to a second flexible substrate 102B and removing the transfer film 202 from the second electrode layer 106B. The transfer adhesive 212 is configured to adhere the non-conductive layer 104B to the second flexible substrate 102B to form the second flexible laminate 116B.

The method 300 of FIG. 3 then further comprises depositing a non-electrically conductive material (or an electrically insulating material) onto at least one of: (i) the first electrode layer, (ii) the second electrode layer, and (iii) the pressure sensing layer, to form a spacer layer (330).

For the flexible sensor 100 of FIGS. 1A and 1B, the pressure sensing layer 108 has a non-continuous first design, comprising void regions having no pressure sensing material. In this case, the method 300 comprises printing the non-conductive material onto the first electrode layer 106A according to a second design to form the spacer layer 110, wherein the first design of the pressure sensing material and the second design of the non-conductive material of the spacer layer 110A do not overlap. As such, the non-conductive material of the spacer layer 110A forms a plurality of discrete spacer elements 110A each contained within a void region of the pressure sensing layer 108.

FIG. 11 shows a top view of an example first laminate 116A and pressure sensing layer 108, suitable for the flexible pressure sensor 100 of FIGS. 1A to 1B. The pressure sensing layer 108 is printed according to a first design, wherein the first design comprises a first region of pressure sensing material 502 bound by a periphery 506 and a plurality of void regions 504 distributed within the first region 502. The void regions 504 do not comprise any pressure sensing material. In the example shown, the spacer layer 110 comprises a plurality of spacer structures 110A. A portion of the spacer structures 110A are arranged within the void regions 504 such that they do not overlap with the pressure sensing material of the first region 502. A second portion of the spacer structures 110A are arranged adjacent to at least a portion of a periphery 506 of the first region 502 of the pressure sensing layer 108.

By contrast, for the flexible sensor 200 of FIGS. 2A and 2B, the non-conductive material of the spacer layer 110 is printed directly onto the pressure sensing layer 108. The non-conductive material is printed according to a design such that the spacer layer 110 comprises a plurality of apertures 110B, exposing the pressure sensor layer 108. Alternatively, a prefabricated mesh of non-conductive material may be deposited directly onto the pressure sensing layer 108 to form the spacer layer 110, or a layer of non-conductive layer may be deposited directly onto the pressure sensing layer and subsequently patterned, for example by UV patterning of UV-curing polymers, to form a non-continuous layer.

Finally, the method 300 comprises attaching the first flexible laminate 116A to the second flexible laminate 116B to form a flexible sensor (340), wherein the pressure sensing layer 108 and the second electrode layer 106B are separated by the spacer layer 110.

In the example described herein, the non-conductive material of the spacer layer 110 is an adhesive material, such as an adhesive polymer. The spacer layer 110 may therefore have dual functionality, both being configured to space apart the pressure sensing layer 108 and the second electrode layer 106B, and to adhere the first flexible laminate 116A to the second flexible laminate 116B. Thus, attaching the first flexible laminate 116A to the second flexible laminate 116B comprises adhering the laminates 116A and 116B by the adhesive spacer layer 110. As such, the spacer layer 110 may also be referred to as an adhesive layer.

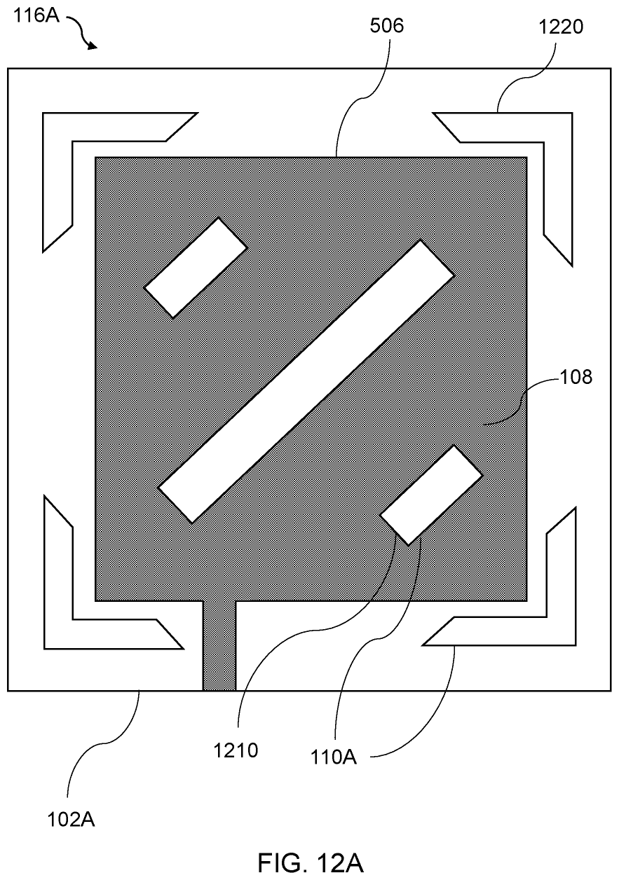

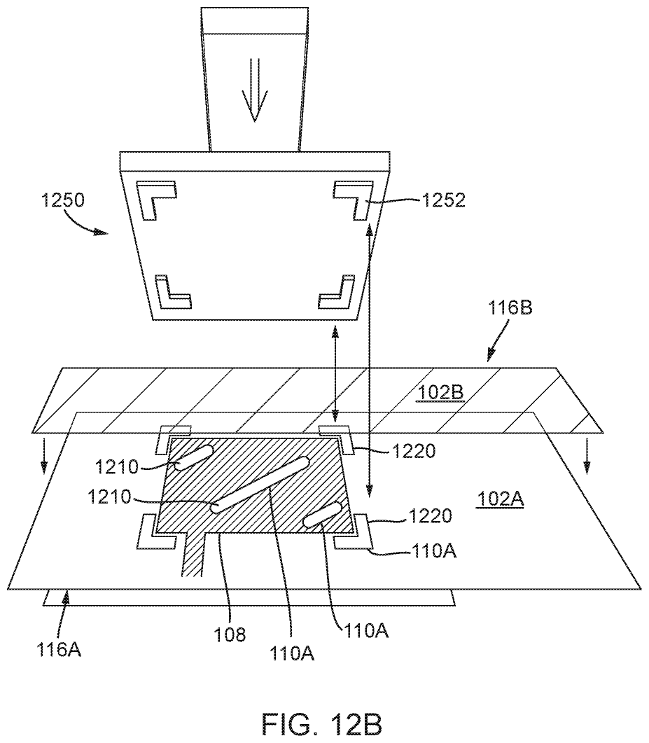

The first flexible laminate 116A and the second flexible laminate 116B may be adhered by the spacer layer 110 by application of at least one of pressure, heat, or UV light, to cure the adhesive. For example, FIG. 12A shows an example top view of a first flexible laminate 116A. The flexible substrate 102A comprises a pressure sensing layer 108 and a plurality of spacer elements 110A, wherein the plurality of spacer elements 110A are printed according to a design. A first portion 1210 of the spacer elements 110A are printed directly onto the pressure sensing layer 108, and a second portion 1220 are printed directly onto the flexible substrate 102A arranged around the periphery 506 of the pressure sensing layer 108. The flexible laminate 116A also comprises at least a non-conductive layer 104A and an electrode layer 106A, however these are arranged underneath the pressure sensing layer 108 and are therefore not shown. FIG. 12B shows the first flexible laminate 116A of FIG. 12A being adhered to a second flexible laminate 116B using a targeted heat press 1250. The second flexible laminate 116B comprises at least a second electrode layer 106B and a second non-conducting layer 104B (not shown). The targeted heat press 1250 comprises localised press plates 1252, wherein the localised press plates 1252 are configured to align with at least a portion of the spacer layer 110. In the example shown, the localised press plates 1252 are configured to align with the second portion 1220 of spacer elements 110A which are printed onto the first flexible substrate 102A. The localised press plates 1252 are configured to apply heat and pressure to this second portion 1220 of spacer elements 110A. In use, the first and second laminates 116A and 116B are aligned as desired such that the spacer layer 110 is arranged between the pressure sensing layer 108 and the second electrode layer 106B (not shown). The localised press plates 1252 and aligned laminate stack, comprising the first and second laminates 116A and 116B, are then aligned and moved to contact one another. The localised press plates 1252 apply heat and pressure to the localised regions of the sensor, including the second portion 1220 of spacer elements 110A. This causes the adhesive spacer structures 110A in the second region 1220 to adhere to the second laminate 116B and cure. However, the portion of spacer elements 110A to which heat and pressure are not applied (i.e., in this example the first portion 1210 of spacer elements 110A) do not adhere to the second laminate 116B. Thus, the second portion 1220 of spacer elements 110A provide adhesive structures, whereas the first portion 1210 of spacer elements 110A are not adhesive and are configured to be purely spacer elements. The skilled person will understand that where UV curing adhesives are used, the targeted heat press 1250 may be substituted for application of UV light through targeted UV templates, wherein the UV templates are configured to permit the incidence of UV light to targeted portions of the spacer layer 110 such that it cures, whilst shielding other portions of the spacer layer 110 from UV light. UV curing may be advantageous to facilitate non-uniform curing.

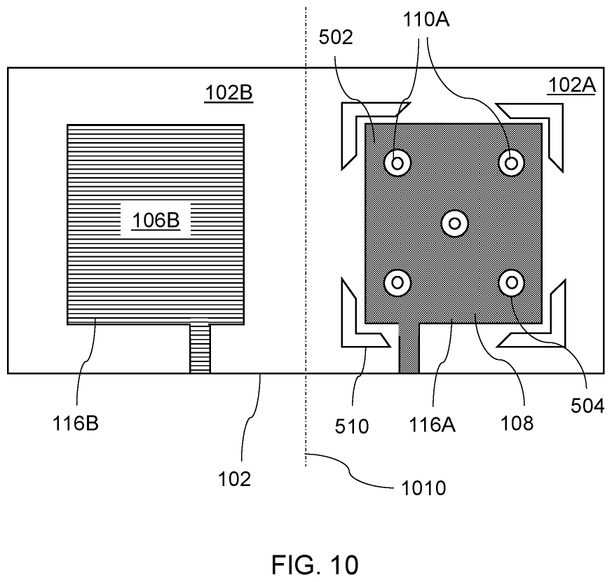

In the example shown in FIG. 10, the first flexible laminate 116A and the second flexible laminate 116B are deposited on a single flexible substrate 102, i.e., wherein the first flexible substrate 102A and the second flexible substrate 102B are different portions of the same flexible substrate 102. Attaching the first flexible laminate 116A to the second flexible laminate 116B to form the flexible sensor then comprises folding the flexible substrate 102 along a fold line 1010 to align the first and second flexible laminates 102A and 102B, where they can then be attached, for example by the adhesive within the spacer layer 110 as described above.

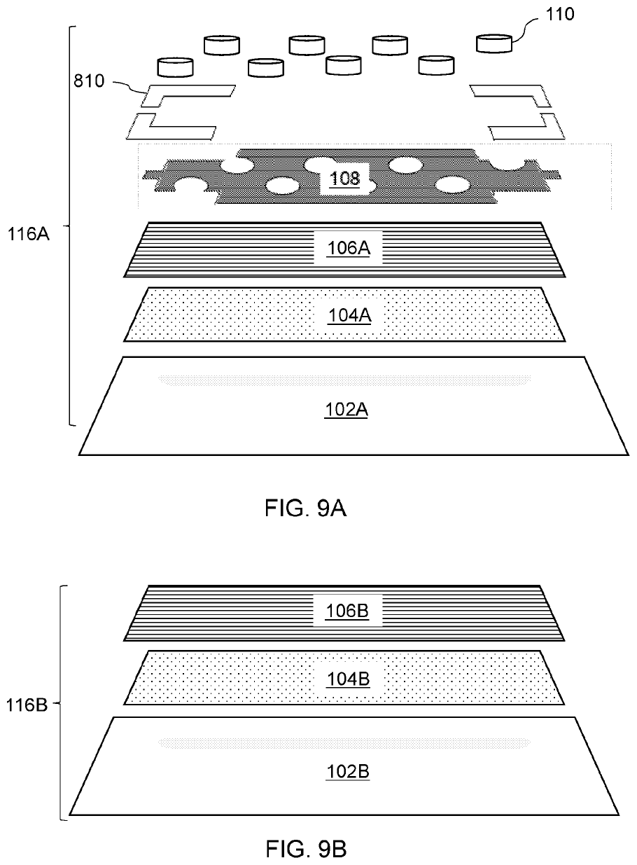

Whilst attachment of the first and second laminates 116A and 116B is discussed with reference to adhesive, in particular wherein the adhesive is provided by the spacer layer 110, the skilled person will understand that alternatively, or in addition, the first and second laminates 116A and 116B may be attached by any suitable means, for example but not limited to using an adhesive separate to the spacer layer 110, sewing, or other mechanical fastenings. For example, FIG. 9A shows an alternative example of a first flexible laminate 116A comprising an adhesive layer 810 separate to the spacer layer 110, wherein the adhesive layer 810 is a different material to the spacer layer 110. Both the adhesive layer 810 and the spacer layer 110 are arranged on the upper surface of the flexible laminate 116A, however they do not overlap. In the example shown, the adhesive layer 810, the spacer layer 110, and the pressure sensing layer 108 are all configured to be printed in a common plane without overlap of any material between the layers. This enables each of the pressure sensing layer 108, the adhesive layer 810, and the spacer layer 110 to perform its function independently to the others without interference. The first and second flexible laminates 116A and 116B are then configured to be adhered together by the adhesive layer 810 whilst the spacer layer 110 is configured to space apart the second electrode layer 106B and the pressure sensing layer 108.

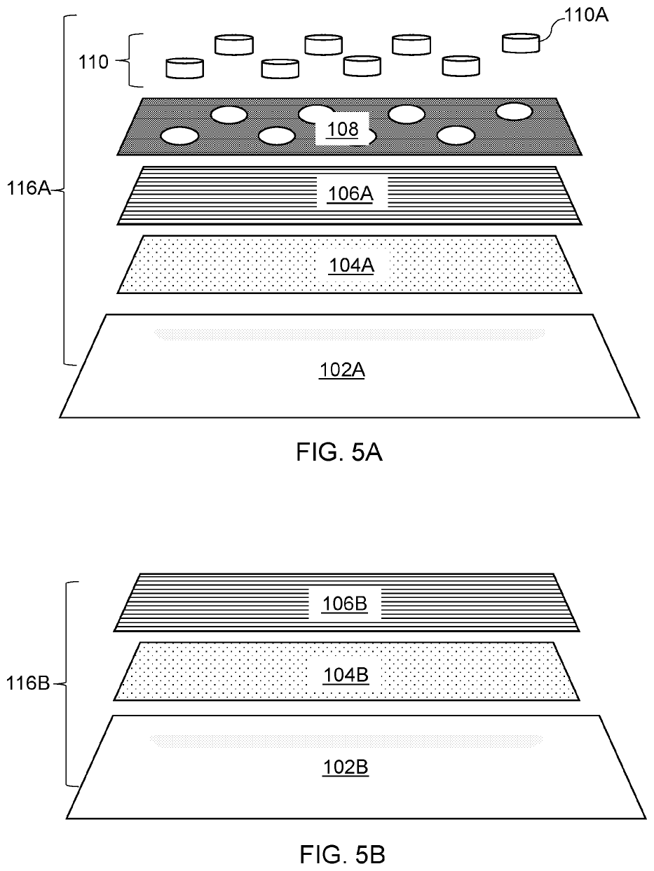

In the example discussed above with reference to the method 300 of FIG. 3, obtaining the first and second flexible laminates 116A and 116B (310; 320) is disclosed with reference to obtaining the laminates 116A and 116B by way of conductive transfer. However, the skilled person will understand that any other suitable method may be used, for example wherein each of the first and second flexible laminates 116A and 116B are obtained by direct printing onto the first or second flexible substrate 102A or 102B, respectively. An example of such is shown in FIG. 5A wherein the first flexible laminate 116A is obtained by printing a non-conductive ink onto a flexible substrate 102A to produce a non-conductive layer 104A, printing an electrically conductive ink onto the non-conductive layer 104A to form the first electrode layer 106A, and printing a pressure sensing ink onto the first electrode layer 106A to produce a pressure sensing layer 108. The second flexible laminate 116B, as shown in FIG. 5B, may be obtained by direct printing onto a second flexible substrate 102B or by way of conductive transfer. The skilled person will understand that the first flexible laminate 116A and the second flexible laminate 116B may be obtained by different methods, for example wherein the second laminate 116B is obtained by way of conductive transfer, and wherein the first laminate 116A is obtained by a direct printing method, and vice versa.

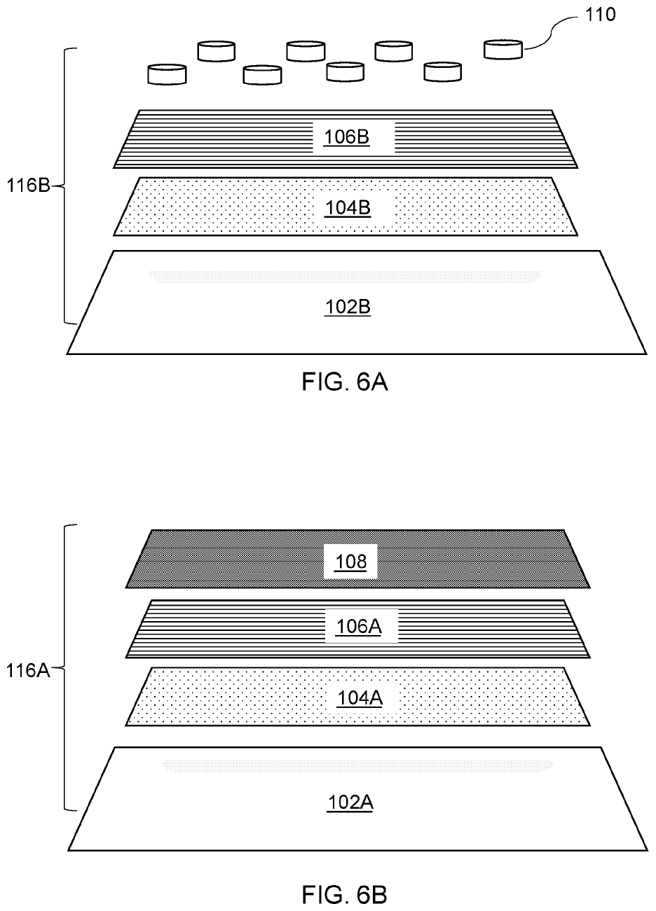

Whilst the examples discussed in relation to sensors 100 and 200 of FIGS. 1A-1B and 2A-2B respectively both disclose depositing the spacer layer 110 onto the first flexible laminate 116A, the skilled person will also understand that in alternative embodiments the non-conductive material of the spacer layer 110 may instead be deposited onto the second flexible laminate 116B. For example, FIG. 6A. illustrates an example second flexible laminate 116B wherein the spacer layer 110 is printed onto the second electrode layer 106B. FIG. 6B illustrates a corresponding first flexible laminate 116A comprising the pressure sensing layer 108. As such, the first flexible laminate 116A and the second flexible laminate 116B may be assembled and attached such that the pressure sensing layer 108 and the second electrode layer 106B separated by the spacer layer 110.

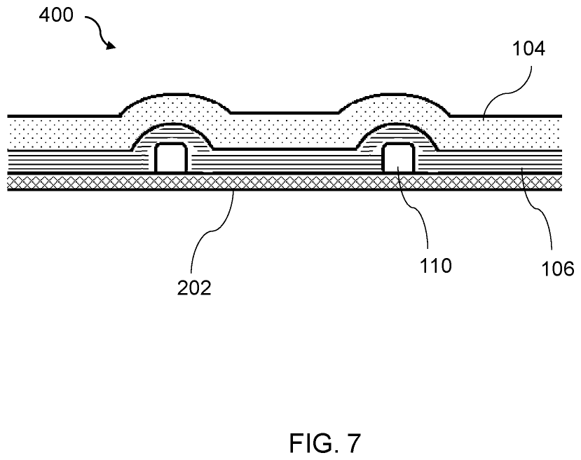

The method 300 is also disclosed above with reference to obtaining the laminate 116A by way of conductive transfer, and subsequently depositing the spacer layer 110 onto the first flexible laminate 116A. However, alternatively, the skilled person will understand that the spacer layer 110 could be incorporated into the conductive transfer 400. In such examples, the spacer layer 110 would be the first layer deposited onto the transfer film 202, wherein the subsequent layers are then deposited on top of the spacer layer 110 in the usual way, for example, the pressure sensing layer 108, followed by the electrode layer 106, the non-conductive layer 104, and the transfer adhesive layer 212. Despite this, printing the spacer layer 110 onto one of the flexible laminates 116A or 116B may be advantageous compared to incorporating the spacer layer 110 within the conductive transfer 400 to minimise warping or undulation of the layers subsequently deposited onto the spacer layer 110 within the conductive transfer 400, as illustrated by the cross-section of conductive transfer 400 in FIG. 7.

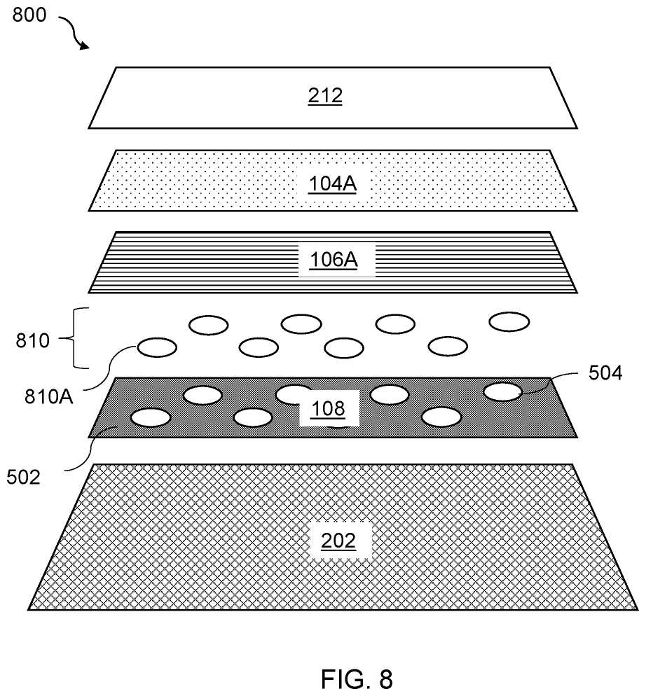

FIG. 8 shows an alternative example conductive transfer 800 comprising a pressure sensing layer 108 printed onto a transfer film 202 in pressure sensing material, a first electrode layer 106A printed onto the pressure sensing layer 108, a non-conductive layer 104A printed onto the first electrode layer 106A, and a transfer adhesive layer 212 printed onto the non-conductive layer 104A. The conductive transfer 800 additionally comprises a plurality of adhesive structures 810A printed onto the transfer film 202 to form a non-continuous adhesive layer 810, wherein the adhesive structures 810A are intermittently spaced and do not overlap with the pressure sensing material of the pressure sensing layer 108. In this example, the pressure sensing layer 108 is printed according to a first design comprising a region of pressure sensing material 502, and a plurality of distributed void regions 504 within the region of pressure sensing material 502, without pressure sensing material. The plurality of adhesive structures 810A are arranged within void regions to avoid overlapping with the pressure sensing material. The depth of the adhesive structures 810A is preferably substantially equal to the depth of the pressure sensing layer 108 to avoid a variation of the undulating effect shown in FIG. 7. The transfer adhesive layer 212 is configured to attach the conductive transfer 800 to a first flexible substrate 102A, as described above, by adhering the insulating layer 104A to the first flexible substrate 102A to form the first flexible laminate 116A. The additional adhesive layer 810 is configured to attach the first flexible laminate 116A to a second flexible laminate 116B (not shown) to form a flexible sensor. In some examples, the adhesive layer 810 may be selectively activated, for example using heat or pressure, as described in more detail in relation to FIGS. 12A and 12B above.

In the context of the present disclosure other examples and variations of the apparatus and methods described herein will be apparent to a person of skill in the art.

1. A flexible pressure sensor comprising:
a first electrode layer and a second electrode layer;
a pressure sensing layer comprising a pressure sensing material, the pressure sensing layer positioned between the first electrode layer and the second electrode layer; and
a spacer layer positioned between the first electrode layer and the second electrode layer, configured to separate the pressure sensing layer and the second electrode layer.

2. The flexible pressure sensor of claim 1, further comprising a first flexible substrate and a second flexible substrate, wherein the first electrode layer and the second electrode layer are positioned between the first flexible substrate and the second flexible substrate.

3. The flexible pressure sensor of claim 2 wherein at least one of the first flexible substrate and the second flexible substrate is a textile layer.

4. The flexible pressure sensor of claim 2, further comprising a first non-conductive layer and a second non-conductive layer, the first non-conductive layer and the second non-conductive layer being positioned between the first flexible substrate and the second flexible substrate, such that the first electrode layer and the second electrode layer are positioned between the first non-conductive layer and the second non-conductive layer.

5. The flexible pressure sensor of claim 1, wherein the spacer layer comprises a plurality of apertures, wherein each aperture is configured to allow the second electrode layer to contact the pressure sensing layer through the aperture under applied pressure.

6. The flexible pressure sensor of claim 5 wherein at least one of (i) aperture size and (ii) spacing between the plurality of apertures is varied across the spacer layer such that pressure sensitivity is configured to be varied across the flexible pressure sensor.

7. (canceled)

8. The flexible pressure sensor of claim 1, wherein the spacer layer is positioned at least partially in a plane of the pressure sensing layer.

9. The flexible pressure sensor of claim 8 wherein the spacer layer has a depth greater than the depth of the pressure sensing layer.

10. The flexible pressure sensor of claim 1, wherein the spacer layer is a non-conductive material.

11. (canceled)

12. The flexible pressure sensor of claim 1, wherein the spacer layer comprises an adhesive configured to adhere the spacer layer between the first electrode layer and the second electrode layer.

13. (canceled)

14. The flexible pressure sensor of claim 12, wherein the adhesive is a heat activated adhesive.

15. The flexible pressure sensor of claim 1, wherein the spacer layer comprises a plurality of spacer structures.

16. (canceled)

17. The flexible pressure sensor of claim 15 wherein the pressure sensing layer comprises at least one region of pressure sensing material, and at least one void region without pressure sensing material, wherein at least one spacer structure of the plurality of spacer structures is bound within the at least one void region.

18. The flexible pressure sensor of claim 1, wherein at least a portion of the spacer layer is arranged adjacent to at least a portion of a periphery of the pressure sensing layer.

19. The flexible pressure sensor of claim 1, wherein the pressure sensing material is configured to vary its resistance as a function of applied force or pressure.

20. A method of producing a flexible sensor, comprising the steps of:
obtaining a first flexible laminate comprising a first flexible substrate, a first electrode layer and a pressure sensing layer, the pressure sensing layer being arranged on a first side of the first electrode layer, the first side being opposite to the first flexible substrate;
obtaining a second flexible laminate comprising a second flexible substrate, and a second electrode layer, the second electrode layer having a first side opposite to the second flexible substrate and a second side adjacent to the flexible substrate;
depositing a non-conductive material onto at least one of: (i) the first side of the first electrode layer, (ii) the first side of the second electrode layer, or (iii) the pressure sensing layer, to form a spacer layer; and
attaching the first flexible laminate to the second flexible laminate, such that the pressure sensing layer and the second electrode layer are separated by the spacer layer.

21. (canceled)

22. The method of claim 20, wherein obtaining the first flexible substrate comprises:
printing a non-conductive ink onto the flexible substrate to produce a non-conductive layer;
printing a second electrically conductive ink onto the non-conductive layer to form the first electrode layer, the second electrically conductive ink being a non-pressure sensing ink; and
printing an electrically conductive pressure sensing ink onto the first electrode layer to produce a pressure sensing layer, wherein printing the pressure sensing layer comprises printing the first design in the second electrically conductive ink.

23. The method of claim 20 wherein obtaining the flexible substrate comprises:
printing an electrically conductive pressure sensing ink onto a transfer substrate to produce the pressure sensing layer;
printing a second electrically conductive ink onto the pressure sensing layer to form the first electrode layer, the second electrically conductive ink being non-pressure sensing;
printing a non-conductive ink over the first electrode layer to produce a non-conductive layer; and
printing an adhesive material over the non-conductive layer to produce a second adhesive layer;
attaching the second adhesive layer to the flexible substrate; and
removing the transfer substrate from the pressure sensing layer.

24. (canceled)

25. The method of claim 20, wherein the non-conductive material of the spacer layer is an adhesive material, such that the spacer layer is configured to provide a first adhesive layer.

26. The method of claim 25, wherein the non-conductive material of the spacer layer is a heat sensitive adhesive material, the method further comprising applying targeted heat patterning to only a portion of the flexible sensor, such that only a portion of the spacer layer is configured to form the first adhesive layer.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)
